(19) United States
(12) Patent Application Publication
(10) Pub. No.: US 2007/0254938 A1
(43) Pub. Date: Nov. 1, 2007

(54) Preventive or Therapeutic Agent for Vascular Intimal Proliferative Disease

(76) Inventors: Kiyoshi Ichikawa, Nagano (JP); Keiko Misawa, Nagano (JP); Shuichiro Hamano, Tokyo (JP)

Correspondence Address: FRENKEL & ASSOCIATES, 3975 UNIVERSITY DR., STE. 330, FAIRFAX, VA, 22030, US

(21) Appl. No.: 11/630,874

(22) PCT Filed: Jun. 29, 2005
(86) PCT No.: PCT/JP05/11896

§ 371 Date: Dec. 26, 2006

(30) Foreign Application Priority Data
Jul. 1, 2004 (JP) 2004-195421

(51) Int. Cl. A61K 31/40; A61P 7/00

(52) U.S. Cl. 514/412

(57) ABSTRACT

The present invention provides pharmaceutical compositions for the prevention or treatment of diseases associated with intimal hyperproliferation of a blood vessel such as restenosis after percutaneous coronary intervention or the like. Moreover, a pharmaceutical composition for the prevention or treatment of diseases associated with intimal hyperproliferation of a blood vessel such as restenosis after percutaneous coronary intervention or the like, which comprises as an active ingredient (2S)-2-benzyl-3-(cis-hexahydro-2-isoindolinylcarbonyl)propionic acid (the generic name: mitiglinide) represented by the following formula or a pharmaceutically acceptable salt thereof, or a hydrate thereof is provided.

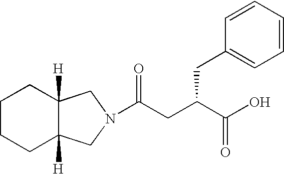

### TECHNICAL FIELD

The present invention relates to pharmaceutical compositions for the prevention or treatment of diseases associated with intimal hyperproliferation of a blood vessel, especially restenosis after coronary artery bypass grafting or percutaneous coronary intervention and the like in the treatment of ischemic heart diseases, comprising as an active ingredient mitiglinide (the chemical name: (2S)-2-benzyl-3-(cis-hexahydro-2-isoindolinylcarbonyl)propionic acid) represented by the chemical structure: 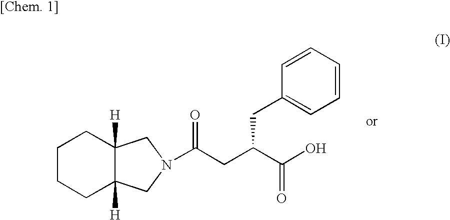
pharmaceutically acceptable salts thereof, or hydrates thereof.

### BACKGROUND ART

Heart disease is one of the most frequent causes of death along with malignant neoplasm, and approximately a half of the heart diseases is caused by is chemic heart diseases such as angina pectoris, cardiac infarction or the like. Therefore, the prevention and treatment of these ischemic heart diseases is getting more important. In the treatment of is chemic heart diseases, coronary artery bypass graft surgery (optionally. hereinafter referred to as CABG) that is a surgical therapy, and percutaneous coronary intervention (optionally hereinafter referred to as PCI) that is a less-invasive therapy play important roles. CABG is a method of revascularization procedures on an occluded blood vessel, which is to form a collateral vessel to bypass the occluded part. For example, off-pump CABG using an internal thoracic artery, a radial artery, a gastroepiploic artery or the like, and others are illustrated. On the other hand, PCI is a general term for methods and devices, which include percutaneous transluminal coronary angioplasty (optionally hereinafter referred to as PTCA) that is a method to dilate a stenosis part of coronary artery using a balloon catheter, and other shaving been newly developed or improved there after. PCT is estimated to be performed in 150,000 cases a year (non-Patent Reference 1). Now, PTCA is also called POBA (plain old balloon angioplasty) in some cases in distinction from new therapeutic devices and methods such as coronary stent, directional coronary atherectomy (optionally hereinafter referred to as DCA), rotational atherectomy (optionally herein after referred to as rotablator) and the like.

The complications that have been problems from the past in the PCI therapy are acute coronary occlusion happening several hours after the operation and restenosis occurring within 6 months after the operation. In such restenosis, it is considered that the narrowing of vascular lumen results mainly from the new intimal hyper proliferation of blood vessel caused by various surgical damages on the vessel wall (non-Patent Reference 2). Even in the coronary stent-method considered to cause that the least occurrence of restenosis in PCI, the present situation is that restenosis is observed in about 20 to 40% of patients depending on surgical techniques and conditions of diseases. Once restenosis occurs, re-operation with a balloon catheter, excision, adding implantation of a coronary stent or the like is needed, and that will impose a heavy burden on not all the patients but also the medical economy.

As the pharmacotherapy for the diseases associated with intimal hyperproliferation of a blood vessel such as restenosis after CABG, PCI or the like, anti-allergic drugs such as tranilast, antithrombotic drugs such as cilostazol, angiotensin II receptor antagonists, angiotensin converting enzyme inhibitors, chymase inhibitors, probcol, trapidil and the like have been studied. And also, the drug-eluting stents coated with sirolimus or paclitaxel have been put to practical use.

Mitiglinide calcium hydrate (the chemical name: bis{(2S)-2-benzyl-3-(cis-hexahydro-2-isoindolinylcarbonyl)-propionic acid}calcium dihydrate) is a commercially available rapid-acting non-SU anti diabetic drug represented by the following formula (II), which is known to correct non-fasting or fasting hyperglycemic states and be useful for the glycemic control in type-II diabetic patients (Patent references 1 and 2). It is also reported to be useful for the prevention or inhibition of progression of diabetic complication due to the glycemic control (Patent reference 3). However, it has not been reported that mitiglinide or a pharmaceutically acceptable salt thereof, or a hydrate thereof is useful for the prevention or treatment of a disease associated with intimal hyperproliferation of a blood vessel.

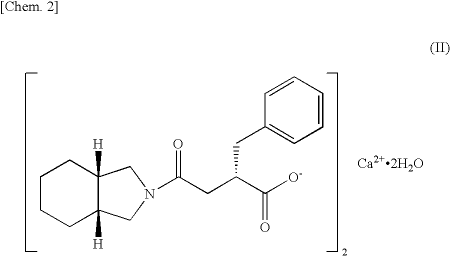

As mentioned above, it has neither ever been known that mitiglinide or a pharmaceutically acceptable salt thereof, or a hydrate thereof is useful for the prevention or treatment of a disease associated with intimal hyperproliferation of a blood vessel including restenosis after CABG, PCI or the like nor suggested in the above references.
Patent Reference 1: Japanese Patent Publication H4-356459;
Patent Reference 2: International Publication WO2004/002473 pamphlet;
Patent Reference 3: International Publication WO2004/002474 pamphlet;
Patent Reference 4: Japanese Patent Publication H6-340622;
Patent Reference 5: Japanese Patent Publication H6-340623;
Non-patent Reference 1: Shigeru Saito, NAIKA (Internal medicine), 2004, Vol. 93, No. 5, pp. 805-811;
Non-patent Reference 2: Toshihiro Tamura et. al., NAIKA (Internal medicine), 2004, Vol. 93, No. 5, pp. 890-896.

### Disclosure of the Invention

Problem to be Solved by the Invention

The object of the present invention is to provide pharmaceutical compositions for the prevention or treatment of a disease associated with intimal hyperproliferation of a blood vessel, for example, restenosis after CABG, PCI or the like.

Means of Solving the Problems

The present inventors have studied earnestly to find an agent effective for intimal hyperproliferation of a blood vessel, and found that mitiglinide calcium hydrate represented by the above formula (II) has a remarkable inhibitory effect on the intimal hyperprlasia caused by scraping endothelium of common carotid artery in rabbit and is useful for the prevention or treatment of a disease associated with intimal hyperproliferation of a blood vessel, thereby forming the basis of the present invention.

The present invention relates to:

[1] a pharmaceutical composition for the prevention or treatment of a disease associated with intimal hyperproliferation of a blood vessel, which comprises as an active ingredient mitiglinide represented by the above formula (I) or a pharmaceutically acceptable salt thereof, or a hydrate thereof;

[2] a pharmaceutical composition as described in the above [1] wherein the disease associated with intimal hyperproliferation of a blood vessel is restenosis after coronary artery bypass grafting or percutaneous coronary intervention;

[3] a pharmaceutical composition as described in the above [1] or [2], which comprises as the active ingredient a calcium salt of mitiglinide represented by the above formula (I) or a hydrate thereof;

[4] a pharmaceutical composition as described in any one of the above [1] to [3], which comprises combination with at least one member selected from a group consisting of an anti-allergic drug, an antiplatelet drug, an angiotensin converting enzyme inhibitor, an angiotensin-II receptor antagonist, a chymase inhibitor and a cell-growth inhibitor; and the like.

The present inventors examined the inhibitory effect of mitiglinide calcium hydrate represented by the above formula (II) on intimal hyperplasia using a model of intimal hyperplasia of a blood vessel caused by scraping endothelium of common carotid artery in rabbit diabetes model. As a result, surprisingly, the present compound exerted significantly inhibitory effect on intimal hyperplasia of a blood vessel compared with the control group at dosage not exerting influence on fasting blood glucose level.

Based on the results, the present compound has a strong inhibitory effect on intimal hyperplasia of a blood vessel and is extremely useful for the prevention or treatment of a disease associated with intimal hyperproliferation of a blood vessel.

The diseases associated with intimal hyperproliferation of a blood vessel include, for example, restenosis after coronary artery bypass graft surgery (CABG), percutaneous coronary intervention (PCI) and the like. As examples of CABG, low invasive CABG such as off-pump CABG, CABG without using artificial heart lung or the like, and bypass surgery using artificial heart lung and stopping the heart and the like can be illustrated, in which blood vessels such as internal thoracic artery, a radial artery, a gastoepiploic artery or the like can be used. As examples of PCI, balloon dilation surgery such as PTCA, cutting balloon or the like, coronary stent including drug-eluting,stent coated with sirolimus, paclitaxel or the like, resection of plaque such as DCA, rotablator or the like, trans-radial coronary intervention (TRI) and the like can be illustrated.

An active ingredient, mitiglinide represented by the above formula (I) or a pharmaceutically acceptable salt thereof, or a hydrate thereof (hereinafter referred to as the compound of the present invention) can be easily prepared according to method described in literature or an analogous method thereof (for example, see the above Patent References. 1, 4 and 5).

As the pharmaceutically acceptable salt of a compound represented by the above formula (I), for example, a salt with an inorganic base such as sodium salt, potassium salt, calcium salt or the like, a salt with an organic amine or amino acid such as morpholine, piperidine, phenylalaninol or the like can be illustrated. A calcium salt is preferable. In addition, as an active ingredient of the present invention, a calcium salt of mitiglinide represented by the above formula (I) or a hydrate thereof is more preferable, and mitiglinide calcium hydrate represented by the above formula (II) is the most preferable.

When the pharmaceutical compositions of the present invention are employed in the practical treatment, various dosage forms are used depending on their uses. As examples of the dosage forms, powders, granules, fine granules, dry syrups, tablets, capsules, injections, solutions, ointments, suppositories, poultices and the like are illustrated, which are orally or parenterally administered.

The pharmaceutical compositions of the present invention can be prepared by suitably admixing with or by diluting and dissolving with an appropriate pharmaceutical additive pharmaceutically used depending on the dosage form such as excipients, disintegrators, binders, lubricants, diluents, buffers, isotonicities, antiseptics, moistening agents, emulsifiers, dispersing agents, stabilizing agents, dissolving aids and the like, and formulating the mixture in accordance with conventional methods. In the case of the uses in combination with other drug(s), they can be prepared by formulating each active ingredient together or individually in a similar manner as defined above.

For example, powders can be formulated by, if desired, admixing well a compound of the present invention with appropriate excipients, lubricants and the like.

For example, tablets can be easily prepared in a method described in literature or an analogous method (see the above Patent References 2 and 3). The tablets, further if desired, can be suitably coated to provide film-coated tablets, sugar-coated tablets, enteric-coated tablets and the like.

For example, capsules can be formulated by, if desired, admixing well a compound of the present invention with appropriate excipients, lubricants and the like and filling it in appropriate capsules. Furthermore, it is also applicable to formulate granules or fine-powders in accordance with conventional methods, and then fill the compositions in capsules.

Furthermore, the pharmaceutical compositions of the present invention can be also used optionally in combination with other drug(s) having an inhibitory effect on intimal hyperproliferation of a blood vessel. Examples of the other drug which can be used in combination with include an anti-allergic drug such as tranilast, pemirolast or the like, an antiplatelet drug such as atropine, cilostazol, ticlopidine, clopidogrel, nafagrel, abciximab, eptifibatide, tirofiban, gantofiban or the like, an angiotensin converting enzyme inhibitor such as imidapril hydrochloride, lisinopril or the like, an angiotensin-II receptor antagonist such as valsartan, losartan potassium, irbesartan or the like, a chymase inhibitor, a cell-growth inhibitor such as sirolimus, paclitaxel, rapamycin or the like, probucol, trapidil, BO-653 and the like.

In the case of uses of the compound of the present invention in combination with the above one or more other drugs, the present invention includes either dosage form of simultaneous administration as a single preparation or separated preparations in way of the same or different administration route, and administration at different dosage intervals as separated preparations in way of the same or different administration route.

When the pharmaceutical compositions of the present invention are employed in the practical treatment, the dosage of a compound of the present invention as the active ingredient is appropriately decided depending on the body weight, age, sex presence or absence of complication, and degree of diseases or treatment of each patient or the like, and as a single dose per adult to be administered orally, which is approximately within the range of from 1 to 60 mg. As an administration method, the drug can be administered 1 to 3 times a day orally or parenterally. Also, in the case of the uses in combination with the above other drug(s), the dosage of the compound of the present invention can be decreased depending on the dosage of the other drug(s)

Effect of the Invention

The compounds of the present invention have an excellent inhibitory effect on intimal hyperplasia of a blood vessel. Therefore, the present invention can provide a pharmaceutical composition useful for the prevention or treatment of a disease associated with intimal hyperproliferation of a blood vessel.

### BRIEF DESCRIPTION OF DRAWINGS

FIG. 1 It is a graph showing effect on intimal hyperplasia after scraping endothelium in alloxan-induced diabetic rabbits. In the figure, the vertical axis indicates the ratio of intimal area to medial area (%). The horizontal axis indicates the normal group, the control group and the mitiglinide-treated group (M group) from the left side.

FIG. 2 It shows effect on blood glucose at 2 weeks after administration of alloxan in alloxan-induced diabetic rabbits. In the figure, the vertical axis indicates the plasma glucose level (mg/dL). The horizontal axis indicates the normal group, the control group and the mitiglinide-treated group (M group) from the left side.

FIG. 3 It shows effect on blood glucose at 6 weeks after administration of alloxan in alloxan-induced diabetic rabbits. In the figure, the vertical axis indicates the plasma glucose level (mg/dL). The horizontal axis indicates the normal group, the control group and the mitiglinide-treated group (M group) from the left side.

### BRIEF MODE TO PRACTICE THE INVENTION

The present invention is further illustrated in more detail by way of the following Example. However, the present invention is not limited thereto.

### EXAMPLE 1

Effect on Intimal Hyperplasia of a Blood vessel Caused by Scraping Endothelium of Common Carotid Artery in Rabbits

Diabetic model animals made by administrating alloxan (50 mg/kg) into Japanese white rabbits (body weight: 2.0 to 3.3 kg) were divided into 2 groups; one was a mitiglinide-treated group (n=5) in which mitiglinide calcium hydrate (3 mg/kg) suspended in 0.5% carboxymethylcellulose was administered orally once a day before feeding for 6 weeks from the next day of alloxan-administration to the day before experimental termination, and the other was the control group (n=4) in which 0.5% carboxymethylcellulose was administered in the same way. Two weeks after the alloxan-administration, the endothelium of the right common carotid artery was scraped using a balloon catheter, and then, the incision on the vessel wall was sutured with care not to cause stenosis. The left common carotid artery was used as a side of Sham-operation (the normal group). Four (4) weeks after the operation of scraping endothelium of a blood vessel, the bilateral common carotid arteries were removed, and the intimal, medial and luminal areas of the sections of the arteries were measured using a morphometric analyzer. Fasting plasma glucose levels were measured 2 weeks after the alloxan-administration (on the day of scraping the endothelium) and 6 weeks after the alloxan-administration (on the day of removing the arteries.

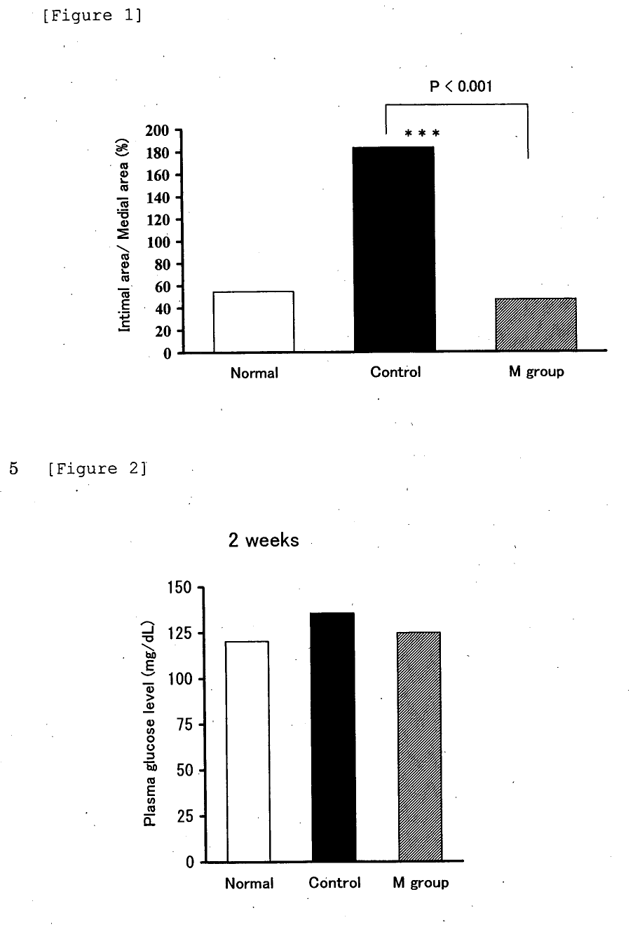

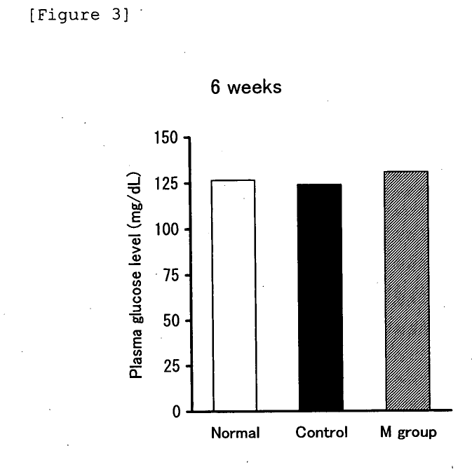

On the intimal hyperplasia caused by scraping endothelium of the common carotid artery in rabbits, the mitiglinide-treated group showed a significant inhibitory effect on the intimal hyperplasia of a blood vessel compared to the control group (FIG. 1). All ratios of the intimal area to medial area on the side untreated by scraping were 0%. Both the mitiglinide-treated and the control groups exerted no influence on the fasting plasma glucose levels at 2 and 6 weeks after alloxan-administration (FIGS. 2 and 3). As mentioned above, it was confirmed that mitiglinide calcium hydrate showed a strong inhibitory effect on the intimal hyperplasia, and no effect on fasting blood glucose was observed in the same experiment.

As mentioned above, it was shown that the pharmaceutical composition of the present invention exerts a significantly excellent inhibitory effect on intimal hyperplasia of the common carotid artery in rabbit and is extremely useful as an agent for the prevention or treatment of restenosis after percutaneous coronary intervention such as percutaneous transluminal coronary angioplasty (PTCA) or the like in the treatment of ischemic heart diseases or the like.

### INDUSTRIAL APPLICABILITY

The pharmaceutical compositions of the present invention are extremely useful as an agent for the prevention or treatment of diseases associated with intimal hyperproliferation of a blood vessel such as restenosis after coronary artery bypass graft surgery, percutaneous coronary, intervention or the like in the treatment of ischemic heart diseases or the like.

1. A pharmaceutical composition for the prevention or treatment of a disease associated with intimal hyperproliferation of a blood vessel, which comprises as an active ingredient mitiglinide or a pharmaceutically acceptable salt thereof, or a hydrate thereof.

2. A pharmaceutical composition as claimed in claim 1 wherein the disease associated with intimal hyperproliferation of a blood vessel is restenosis after coronary artery bypass grafting or percutaneous coronary intervention.

3. A pharmaceutical composition as claimed in claim 1 or 2, which comprises as an active ingredient a calcium salt of mitiglinide or a hydrate thereof.

4. A pharmaceutical composition as claimed in any one of claims 1 to 3, which comprises combination with at least one member selected from a group consisting of an anti-allergic drug, an antiplatelet drug, an angiotensin converting enzyme inhibitor, an angiotensin-II receptor antagonist, a chymase inhibitor and a cell-growth inhibitor.

5. A method for the prevention or treatment of a disease associated with intimal hyperproliferation of a blood vessel, which comprises administering an effective amount of mitiglinide or a pharmaceutically acceptable salt thereof, or a hydrate thereof.

6. A method for the prevention or treatment as claimed in claim 5 wherein the disease associated with intimal hyperproliferation of a blood vessel is restenosis after coronary artery bypass grafting or percutaneous coronary intervention.

7. A method for the prevention or treatment as claimed in claim 5 or 6 wherein the drug administered is mitiglinide calcium hydrate.

8. A method for the prevention or treatment as claimed in any one of claims 5 to 7, which comprises using in combination with at least one member selected from a group consisting of an anti-allergic drug, an antiplatelet drug, an angiotensin converting enzyme inhibitor, an angiotensin-II receptor antagonist, a chymase inhibitor and a cell-growth inhibitor.

9. A use of mitiglinide or a pharmaceutically acceptable salt thereof, or a hydrate thereof for the manufacture of a pharmaceutical composition for the prevention or treatment of a disease associated with intimal hyperproliferation of a blood vessel.

10. A use as claimed in claim 9 wherein the disease associated with intimal hyperproliferation of a blood vessel is restenosis after coronary artery bypass grafting or percutaneous coronary intervention.

11. A use as claimed in claim 9 or 10 wherein the drug used is mitiglinide calcium hydrate.

12. A use as claimed in any one of claims 9 to 11 which comprises using in combination with at least one member selected from a group consisting of an anti-allergic drug, an antiplatelet drug, an angiotensin converting enzyme inhibitor, an angiotensin-II receptor antagonist, a chymase inhibitor and a cell-growth inhibitor.
